(19) United States
(12) Patent Application Publication
(10) Pub. No.: US 2016/0374648 A1
(43) Pub. Date: Dec. 29, 2016

(54) WEARABLE DEVICE AND METHOD FOR COLLECTING OCULAR FLUID

(71) Applicant: KONINKLIJKE PHILIPS N.V., EINDHOVEN (NL)

(72) Inventors: SIMA ASVADI, EINDHOVEN (NL); KORAY KARAKAYA, EINDHOVEN (NL); RON MARTINUS LAURENTIUS VAN LIESHOUT, GELDROP (NL); MAARTEN PETRUS JOSEPH KUENEN, NOORD BRABANT (NL); KIRAN HAMILTON J. DELLIMORE, UTRECHT (NL)

(21) Appl. No.: 15/194,549

(22) Filed: Jun. 27, 2016

(30) Foreign Application Priority Data
Jun. 26, 2015 (EP) 15173975.2

(51) Int. Cl. A61B 10/00

(52) U.S. Cl. CPC A61B 10/0045; A61B 2010/0067

(57) ABSTRACT

The present invention relates to a wearable device for collecting ocular fluid of a user, comprising a fluid channel for enabling flow of ocular fluid within the fluid channel when the wearable device is worn by the user, the fluid channel extending from an open end in an annular geometry, the open end being configured to receive ocular fluid, wherein the wearable device further comprises one or more modular compartment units detachably connected to the fluid channel and/or include a porous layer comprising a plurality of pores extending through the porous layer in a radial direction, wherein the fluid channel and/or the one or more modular compartment units are configured to contain a hydrophilic material in an inner space of each modular compartment unit, the hydrophilic material being configured to absorb the ocular fluid, wherein the fluid channel and/or the inner space of each modular compartment unit is, when the device is worn by the user, in fluidic connection with the ocular fluid of the user via the open end, the plurality of pores and/or a corresponding fluid inlet of the modular compartment unit.

### CROSS-REFERENCE TO RELATED APPLICATIONS

The present application claims the priority benefit of European Patent Application Number EP 15173975.2, filed Jun. 26, 2015, the entire disclosure of which is hereby incorporated herein by reference.

### FIELD OF THE INVENTION

The present invention relates to a wearable device and method for collecting ocular fluid. It finds applications in tear fluid analysis, in particular for detecting biomarkers, therapeutic drugs as well as monitoring and management of ocular side effects of therapeutic drugs.

### BACKGROUND

Tear fluid, also known as ocular fluid, is a result of lacrimation which is the process of tear secretion. Tear fluid plays a vital role in protecting the ocular surface from environmental hazards as well as invading pathogens. Tear fluid also maintains optimal conditions for ocular health and vision through hydrating and lubricating the ocular surface. Tear fluid is a complex mixture containing soluble and insoluble mucins, proteins and aqueous components covered by an upper lipid layer.

For these properties, tear fluid can be applied in various fields, for instance as a source of biomarkers or as a biomaterial for drug response and disease monitoring.

Regardless of the goal of the investigation and method used in tear fluid analysis, in order to perform any analysis on ocular fluids, a tear sample has to be collected. Tear fluid collection must be performed with minimum stimulation of the eye. This is particularly important as it has been shown that the composition of tear that has been created by mechanical or chemical eye stimulation is different from normally secreted tear fluid.

Current methods for tear fluid collection involve collecting a sample of tear fluid followed by an analysis routine. The tear sample is normally collected by means of tubes, in particular micro-tubes, made out of e.g. glass or silicone, which are held in the so-called “tear pool” for 5 minutes. If the tear samples are generated based on stimulation/irritation of the eye, e.g. by rubbing or nasal stimulation, they are collected outside of the eyes.

Further methods include integrating the tube in a specific device, so that a subsequent analysis can be performed right after sample taking. Another practiced method involves placing an absorbing strip of specific “filter papers” normally with dimensions of 7×40 mm in the lower conjunctiva of the patient's eye after which the patient has to close his eye for 5 minutes while the strip remains in his eye. During this time, tear fluid is collected.

Accurate determination of tear fluid volume is important as the concentration of any detected compound is calculated based on the collected tear volume. The existing methods of tear sample collection have the following shortcomings. First, tear analysis using samples collected by those methods are able to provide information about tear composition at specific time points (“point data”). However, such methods are not able to provide information about tear composition variability over time. Second, devices known in the past for tear fluid collection often create a chance of stimulation of the patient's eye when such devices are brought in contact with the eye surface. Such stimulation can cause tear generation with a different composition from that under normal conditions. Third, the known methods do not provide an easy possibility to collect tear samples during sleeping hours without any inconvenience to the patients and caregivers. Fourth, the known methods are restricted in the collectable tear volume, since under normal conditions each eye contains only 7-10 of tear. This volume is normally decreased for aging people and more significantly so if they suffer from such conditions as “dry eye” that causes a decrease in tear fluid secretion hence making tear fluid collection even more challenging.

US2014/0088381A1 discloses collection of tear fluid both in the structural parts of the contact lens and in the cavities created in the contact lens. However, no mechanism is disclosed for collecting a predetermined volume of tear.

US2014/309554A1 relates to a device for sampling tear fluid that comprises an extraction element adapted to be applied on the eye to draw tear fluid therefrom. The extraction element includes at least one tube and a distal portion with at least one opening. The device further comprises a collection vessel connected to said tube and suction means adapted to continuously draw tear fluid from the eye to the collection vessel through the extraction element.

US2014/343387A1 describes a system for an energized ophthalmic device with a media insert that includes microfluidic elements upon or within the media insert, and which can be used for analyzing an analyte such as glucose in a fluid sample, and/or for administering a medicament to treat an abnormal condition identified during the analyte analysis in the fluid sample.

### SUMMARY OF THE INVENTION

It is an object of the present invention to provide a device and method for collecting ocular fluid of a user which enable analysis of tear fluid composition over time and with higher precision while avoiding undesirable effects due to eye stimulation or irritation. This object is solved by the wearable device for collecting ocular fluid of a user of claim 1 and the method for collecting ocular fluid of a user of claim 14.

In a first aspect of the present invention a wearable device for collecting ocular fluid of a user is provided that comprises a fluid channel for enabling flow of ocular fluid within the fluid channel when the wearable device is worn by the user, the fluid channel extending from an open end in an annular geometry around a revolution axis, the open end of the fluid channel being configured to receive ocular fluid. The wearable device further comprises a porous layer arranged on an external surface of the fluid channel and comprising a plurality of pores extending through the porous layer in a second radial direction, wherein the fluid channel contains a hydrophilic material in a hollow space within the fluid channel, the hydrophilic material being provided in a volume that is smaller than the volume of the hollow space within the fluid channel, wherein said hollow space is, when the device is worn by the user, in fluidic connection with the ocular fluid of the user via the open end and the plurality of pores; and/or one or more modular compartment units detachably connected to at least one of the outer edge and the inner edge on the external surface of the fluid channel, the one or more modular compartment units contain a hydrophilic material in an inner space of each modular compartment unit, the hydrophilic material being provided in a volume that is smaller than the volume of the inner space of the corresponding modular compartment, wherein the inner space of each modular compartment unit is, when the device is worn by the user, in fluidic connection with the ocular fluid of the user via the open end and a corresponding fluid inlet of the modular compartment unit, wherein the hydrophilic material contained in the hollow space within the fluid channel and/or the hydrophilic material contained in the inner space of each modular compartment unit is configured to absorb the ocular fluid and increase in volume in proportion to the amount of ocular fluid absorbed.

In another aspect of the present invention a method for collecting ocular fluid of a user, wherein the method comprises using the wearable device described herein, and comprises the step of: using the fluid channel to enable flow of ocular fluid within the fluid channel when the wearable device is worn by the user, the fluid channel extending from an open end in an annular geometry around a revolution axis, the open end of the fluid channel being configured to receive ocular fluid, wherein the wearable device further comprises a porous layer arranged on an external surface of the fluid channel and comprising a plurality of pores extending through the porous layer in a second radial direction, wherein the fluid channel contains a hydrophilic material in a hollow space within the fluid channel, the hydrophilic material being provided in a volume that is smaller than the volume of the hollow space within the fluid channel, wherein said hollow space is, when the device is worn by the user, in fluidic connection with the ocular fluid of the user via the open end and the plurality of pores; and/or one or more modular compartment units detachably connected to at least one of the outer edge and the inner edge on the external surface of the fluid channel, wherein the one or more modular compartment units contain a hydrophilic material in an inner space of each modular compartment unit, the hydrophilic material being provided in a volume that is smaller than the volume of the inner space of the corresponding modular compartment unit, wherein the inner space of each modular compartment unit is, when the device is worn by the user, in fluidic connection with the ocular fluid of the user via the open end and a corresponding fluid inlet of the modular compartment unit;

wherein the hydrophilic material contained in the hollow space within the fluid channel and/or the hydrophilic material contained in the inner space of each modular compartment unit is configured to absorb the ocular fluid and increase in volume in proportion to the amount of ocular fluid absorbed.

In yet further aspects of the present invention, there are provided a computer program which comprises program code means for causing a computer operatively coupled to a wearable device as disclosed herein to perform the steps of the method disclosed herein on the wearable device when the computer program executed on the computer as well as non-transitory computer-readable recording medium that stores therein a computer program product, which, when executed by a device, causes the method disclosed herein to be performed.

Preferred embodiments of the invention are defined in the dependent claims. It shall be understood that the claimed method and computer program have similar and/or identical preferred embodiments as the claimed wearable device and as defined in the dependent claims.

The fluid channel enables diffusion/flow of ocular fluid when the present device is worn by the user. The fluid channel extends in an annular geometry or format, which means that the fluid channel is a ring-shaped or annular channel covering circumferentially an angle. The angle can be smaller than 360° (in the case of an open annular geometry) or equal to 360° (in the case of a closed annular geometry).

Preferably, the fluid channel has an at least partially tubular cross-section and extends along an annular axis from the open end in an annular geometry around the revolution axis, which is perpendicular to the annular axis.

In such cases, the second radial direction is preferably perpendicular to the annular axis.

The cross-section of the fluid channel may extend circumferentially around the annular axis over an angle that can be equal to 360° or smaller than 360° (i.e., tube sliced along its annular axis), preferably equal to 180° (“half tube”).

In some embodiments the hydrophilic material contained in the hollow space within the fluid channel is the same as the hydrophilic material contained in the inner space of each modular compartment unit, while in some other embodiments these hydrophilic materials are different.

In the context of the present invention, the expression that the volume of a hydrophilic material increases in proportion to the amount of ocular fluid absorbed preferably relates to the fact that the larger the amount of ocular fluid absorbed in the hydrophilic material, the larger the volume of said hydrophilic material. However, the term “in proportion” is not to be construed as necessarily requiring linear proportionality.

For the case that the fluid channel is configured to detachably attach the one or more compartment units, the inner space of each modular compartment unit is in fluidic connection with the ocular fluid flowing on the ocular surface of the user. In this way, the ocular fluid can flow from the ocular surface into the inner space of each modular compartment unit via the corresponding fluid inlet of the modular compartment unit, thereby enabling collecting of ocular fluid.

The one or more compartment units are configured as modular units, so that each compartment unit is a separate unit. In case the compartment region comprises a plurality of modular compartment units, the individual compartment units in combination form a compartment region.

Each single modular compartment unit has an inner space, in which the hydrophilic material can be contained. In particular, the hydrophilic material for each modular compartment unit can be provided in a volume that is smaller than the volume of the inner space of the modular compartment unit. Since each modular compartment unit can be built with an inner space having a predetermined volume, the amount/volume of ocular fluid that can be absorbed by the hydrophilic material contained in the inner space is limited by the predetermined volume of the inner space. In this way, the present wearable device enables to collect ocular fluid of the user with a controlled volume. Advantageously, the concentration of substances contained in the collected ocular fluid can be determined with higher precision, leading to higher reliability of tear analysis.

The one or more modular compartment units are detachably connectable to the fluid channel. This means that the number of the modular compartment units to be connected to the fluid channel can be randomly chosen depending on the user's application. Advantageously, this achieves high application adaptability of the wearable device.

The fluid inlet of the modular compartment unit is in fluidic connection with the fluid channel. The fluid channel is therefore configured to detachably connect the one or more modular compartment units, wherein the fluid channel is in fluidic connection with the ocular fluid of the user, when the device is worn by the user. In this way, the fluid channel enables the fluidic connection between the fluid inlet of the modular compartment unit and the ocular fluid of the user wearing the device. Advantageously, the fluidic connection between the ocular fluid of the user and the individual modular compartment units can be provided more reliably.

Preferably, multiple modular compartment units are arranged serially on a tear inlet path. The tear fluid comes into contact with the modular compartment units in a serial manner. Tear fluid is collected first in a first modular compartment unit which is the one closest to the inlet of the tear inlet path. After the amount of fluid absorbed by the hydrophilic material within the inner space of the first modular compartment unit has saturated, in particular as a result of swelling of the hydrophilic material, the tear collection volume of the first modular compartment unit has saturated. Then, tear fluid will be collected in a second modular compartment unit next to the first modular compartment unit. The same process continues after the tear collection volume of the second modular compartment unit has saturated. Using this process, the tear fluid serially absorbed in the modular compartment units varies over time. Different modular compartment units therefore collect tear fluid during different time intervals.

Additionally or alternatively, the fluid channel can contain or be filled with the hydrophilic material. In this case, the fluid channel itself may absorb ocular fluid, so that the one or more modular compartment units may be omitted.

For the case that the fluid channel includes the porous layer, the fluid channel is in fluidic connection with the ocular fluid of the eye via the plurality of pores. The porous layer can be made of a porous material, in particular a membrane, which comprises a plurality of pores extending through the thickness of the layer in the natural state of the porous material. The plurality of pores extending through the layer in the second radial direction advantageously enable a fluidic connection between the surrounding of the fluid channel and the hollow space within the fluid channel.

The diffusion/transport rate of ocular fluid depends on the size and/or the density of the pores. Hence, with the help of the pores, the wearable device can be configured to collect ocular fluid while enabling to control the volume of the collected ocular fluid with high precision and to provide tear composition variations over time even without the use of hydrophilic materials such as hydrogels. Pore dimensions and density determine the diffusion rate over time for a particular compartment. If the purpose is to collect tear fluid over several time periods porous layers with several pore density and dimensions can be used.

The device is wearable by a user and can be preferably incorporated in a contact lens. The tear collection can be carried out over time. Advantageously, this enables to provide information about tear composition variability over time. Also, the amount for volume of the tear fluid collectable by the wearable device is not restricted to the amount of tear contained in a human eye at a given time, so that the tear analysis can be carried out based on an increased amount of collected tear fluid, leading to higher reliability of tear analysis. Besides reducing or even avoiding undesirable effects of eye stimulation/irritation, the present invention also enables to collect tear sample during sleeping hours without inconvenience to the patient and the caregivers.

In a preferable embodiment, the fluid inlet is closable by the hydrophilic material contained in the corresponding inner space having absorbed a predetermined amount of ocular fluid. After the hydrophilic material has absorbed the predetermined amount of ocular fluid, the hydrophilic material swells and increases in volume. This process continues until the volume of the hydrophilic material reaches the predetermined volume of the inner space of the modular compartment unit. Then, the fluid inlet of the modular compartment unit is closed by the hydrophilic material sealing the fluid inlet from inside of the inner space. In this way, no more tear fluid can enter the inner space of the modular compartment unit. Advantageously, the present wearable device enables a self-actuating closure of the modular compartment unit and consequently a more precise volume determination for the collected tear fluid.

Preferably, the fluid inlet is formed on a deformable side of the modular compartment unit, the deformable side being inwardly curved or sunken before the hydrophilic material contained in the corresponding inner space has absorbed the ocular fluid. Further preferably, the deformable side of the modular compartment unit is planar or outwardly curved after the hydrophilic material contained in the corresponding inner space has absorbed the predetermined amount of ocular fluid. The predetermined volume of the inner space is therefore reached when the deformable side of the modular compartment unit has turned from an inwardly sunken state to a planar or outwardly curved state after the hydrophilic material contained in the corresponding inner space has absorbed the predetermined amount of ocular fluid. Advantageously, the amount of absorbed ocular fluid can be determined with high precision.

Preferably, the fluid channel is a ring-shaped or annular channel comprising an outer edge and an inner edge spaced from the outer edge along a first radial direction perpendicular to the revolution axis. Advantageously, the area enclosed by the ring-shaped channel can be used for receiving incoming light, so that the present wearable device can be built with higher adaptability to the eye of the user. “Ring-shaped” can mean a ring whose outer and/or inner edge covers a spherical angle equal to or smaller than 360° in the circumferential direction.

Further preferably, the one or more modular compartment units are detachably connected to the outer edge or the inner edge on the external surface of the fluid channel. This means that at least one of the modular compartment units can be detachably connected externally to the ring-shaped channel on the outer edge or the inner edge, so that the number of modular compartment units detachably connectable to the fluid channel is increased.

In another preferable embodiment, the one or more modular compartment units, or one or more additional modular compartment units, are detachably connected to the fluid channel in a hollow space within the fluid channel. This embodiment employs advantageously the inner space of the fluid channel to accommodate the one or more modular compartment units.

In another preferable embodiment, the fluid channel comprises a hydrophobic material and/or extends in the annular geometry between an open end for receiving fluid and a closed end. The usage of the hydrophobic material for the fluid channel avoids advantageously chemical interactions between the tear fluid and the fluid channel as well as the absorption of the tear fluid by the fluid channel, so that at least a majority of the ocular fluid entering the fluid channel can be collected by the one or more modular compartment units without change of the composition of the ocular fluid. This advantageously increases the reliability of the tear analysis. The closed end of the of the fluid channel prevents the ocular fluid entering the fluid channel from exiting the fluid channel shortly after entrance, so that the ocular fluid can be collected more easily.

In another preferable embodiment, the one or more modular compartment units comprise a hydrophobic material. Such hydrophobic material prevents chemical interactions between the modular compartment units and the ocular fluid entering the inner space of the modular compartment units as well as absorption of the ocular fluid by the modular compartment unit. Advantageously, at least the majority of the ocular fluid entering the inner space of each modular compartment unit can be absorbed by the hydrophilic material, leading to a more reliable tear fluid collection.

In another preferable embodiment, the hydrophilic material comprises a hydrogel. Hydrogels are materials containing cross-linked polymeric chains, so that the hydrogels are able to absorb aqueous solutions without dissolving. Advantageously, the collection of ocular fluid can be carried out with high security and reliability.

### BRIEF DESCRIPTION OF THE DRAWINGS

These and other aspects of the invention will be apparent from and elucidated with reference to the embodiments described hereinafter. In the following drawings:

FIG. 1 is a schematic illustration of an eye of a human;

FIG. 2 is another schematic illustration of an eye of a human;

FIG. 3A is a schematic illustration of an exemplary micro-tube for tear sample collection, in accordance with an exemplary embodiment of the present invention;

FIG. 3B is an illustration of an exemplary tube for tear sample collection, in accordance with an exemplary embodiment of the present invention;

FIG. 4A is an illustration of an exemplary integratable micro-tube for tear sample collection, in accordance with an exemplary embodiment of the present invention;

FIG. 4B is an illustration of the exemplary integratable micro-tube of FIG. 4A in conjunction with an exemplary analysis device, in accordance with an exemplary embodiment of the present invention;

FIG. 5A is a schematic diagram showing an exemplary cross-link density and the modulus of elasticity of a hydrogel as a function of network concentration, in accordance with an exemplary embodiment of the present invention;

FIG. 5B is an illustration showing the volume swelling ratio of the hydrogel of FIG. 5A as a function of the network concentration, in accordance with an exemplary embodiment of the present invention;

FIG. 6 is a schematic illustration of a network of connected micro-pores of a silicone-hydrogel material, in accordance with an exemplary embodiment of the present invention;

FIG. 7A is an illustration showing schematically an exemplary modular compartment unit for containing a hydrogel, with the modular compartment unit being in an open state, in accordance with an exemplary embodiment of the present invention;

FIG. 7B is an illustration showing schematically the exemplary modular compartment unit of FIG. 7A, with the modular compartment unit being in a closed state, in accordance with an exemplary embodiment of the present invention;

FIG. 8 is an illustration showing schematically an exemplary plurality of modular compartment units detachably connected to a fluid channel, in accordance with an exemplary embodiment of the present invention;

FIG. 9A is an illustration of another exemplary fluid channel comprising a plurality of pores, in accordance with an exemplary embodiment of the present invention; and

FIG. 9B is an illustration of the exemplary fluid channel shown in FIG. 9A in cross section, in accordance with an exemplary embodiment of the present invention.

### DETAILED DESCRIPTION OF THE INVENTION

Tear fluid is a result of lacrimation (i.e. the process of tear secretion) that is driven by lacrimal glands, the accessory lacrimal glands and goblet cells of the conjunctiva, coupled with some fluid permeating from corneal and conjunctival tissue.

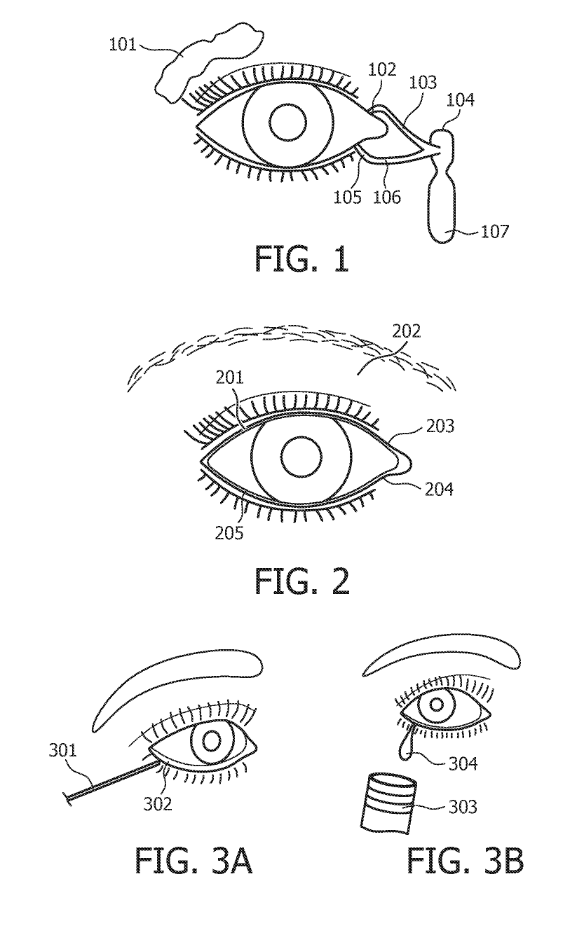

FIG. 1 shows a schematic illustration of a human eye, wherein the lacrimal gland 101, the superior lacrimal punctum 102, the superior lacrimal canal 103, the lacrimal sac 104, the inferior lacrimal punctum 105, the inferior lacrimal canal 106 and the nasolacrimal canal 107 can be seen. FIG. 2 shows schematically another schematic illustration of a human eye, wherein the lacrimal gland 201 (in-sida), the eyelid 202, the lacrimal canaliculi 203 (in lacrimal sac), lacrimal puncta 204 and conjunctiva 205 (adherent to cornea) are shown.

Tear fluid plays a vital role in protecting the ocular surface from environmental hazards as well as invading pathogens. Tear fluid maintains optimal conditions for ocular health and vision through hydrating and lubricating the ocular surface. Tear fluid is a complex mixture containing soluble and insoluble mucins, proteins and aqueous components covered by an upper lipid layer.

Tear fluid contains various molecules including a large variety of proteins. The protein composition of the tear fluid can change with respect to various local and systemic diseases. Tear components show great potential as biomarkers in the development of clinical assays for various human diseases. Furthermore, biomarkers represent promising targets for drug development and can be used to monitor the disease state or treatment responses, and accordingly improve the standards of patient care. Examples for biomarkers are Lactate Dehydrogenase (LDH), α1-antitrypsin, cortisol and melatonin.

Lactate dehydrogenase (LDH) is an enzyme that facilitates the conversion of pyruvate to lactate and vice versa which is of medical significance. Found in tissues such as blood cells and heart muscle, it is released during tissue damage. That is why it can also be used as a marker of metabolic function, tissue oxygenation and find applications in areas related to heart function.

α1-antitrypsin (A1AT) is a peotease inhibitor that protects tissues from enzymes of inflammatory cells. In its absence, neutrophil elastase is free to break down elastin which contributes to the elasticity of the lungs, resulting in respiratory complications such as emphysema, or COPD.

Cortisol is a hormone secreted by adrenal gland in response to physiological and environmental stress and low blood glucose level. Its function is to stimulate glycogenesis and suppress anti-stress and anti-inflammatory path ways and is involved in metabolism of fat, protein and carbohydrates. Prolonged cortisol deregulation has been associated with a variety of conditions such as hypertension, sleep disorder, fatigue, depression and dementia.

Melatonin is the most commonly used marker for measuring the circadian phase position. The onset of melatonin each evening is called the dim-light melatonin onset (DLMO). To assess the circadian phase position numerous time points need to be measured before and during the night.

Tear fluid basically reflects the events in the blood in a similar way to saliva. However, as the eye is a more protected environment compared to the mouth in terms of bacterial activity (e.g. through food and beverages) it is a more “pure” material to work with in terms of accessing bodily response to various diseases and their corresponding treatments. This benefit will be significantly enhanced with the continuous sample collection methods proposed in this invention. Examples of biomarker-disease combinations are provided in the section “Applications of the invention” at the end of the document.

Tear fluid analysis can be applied in monitoring body's response to therapeutic drugs either directly or through their ocular side effects. Multiple classes of therapeutic drugs can be detected in tear fluid by different analytical methods. Examples are therapeutics used in treating psychological disorders (e.g. bi-polar disorder, depression), inflammation, chemotherapy, glaucoma through detection of therapeutic drugs such as, Phenobarbital, Carbamazepine, Methotrexate, etc.

Many therapeutic drugs create side effects including those related to the eye. One common side effect is the condition known as “dry eye”. Medications such as topical and systemic beta blockers (e.g. Carvedilol), tricyclic anti-depressants and topical non-steroidal anti-inflammatory agents as well as contraceptive pills are among the therapeutic drugs known to be among the causes of ‘dry eye’ condition. Symptoms of dry eye are increased itchiness and stinging sensation in the eyes as well as hyper-osmolarity caused by increased rate of evaporation and/or decreased rate of tear secretion which leads to a more concentrated tear film with a reduced aqueous component leading to increased osmolarity.

Therefore, determination of tear osmolarity is currently used as an objective method to diagnose dry eye condition. A number of existing methods for determining tear osmolarity are listed below. In these methods the total ocular fluid is collected and tested with the aim to make a distinction between “normal” ocular fluid and that of a patient with dry eye in terms of the ocular fluid's aqueous and solid component ratio. These osmolarity methods include: 1) Freezing point depression: method in which freezing point of the ocular fluid with high osmolarity is depressed as particle content has increased; 2) Vapor pressure: in which vapor pressure of the ocular fluid with high osmolarity is lower for the same reason that vapor pressure of a solution is lower than the vapor pressure of the pure solvent. This methods requires a very high samples volume (5 μl); 3) Electrical impedance: Determining the electrical impedance of the ocular fluid leads to a measure of tear osmolarity as decreased water content is reflected in the impedance level.

Regardless of the goal of the investigation and the method used in tear fluid analysis, in order to perform any analysis on ocular fluids a tear sample has to be collected. Tear collection must be performed with minimum stimulation of the eye: this is particularly important as it has been shown that composition of the tear that has been created by mechanical or chemical eye stimulation is different from normally secreted tear. Current methods involve collecting a sample of tear fluid followed by an analysis routine. The tear sample is normally collected by means of (micro-) tubes made out of e.g. glass or silicone, which are held in the so called “tear pool” for 5 minutes. If the tear samples are generated based on stimulation/initiation of the eye e.g. by rubbing or nasal stimulation they are collected outside of the eyes. Two examples are depicted in FIG. 3A, B. In FIG. 3A, a micro-tube 301 is shown for collecting tear fluid of an human eye 302. FIG. 3B shows a similar method using a tube 303 for collecting tear sample 304. Tear samples are often deposited on filter papers, for later isolation, dilution and freezing for storage purposes.

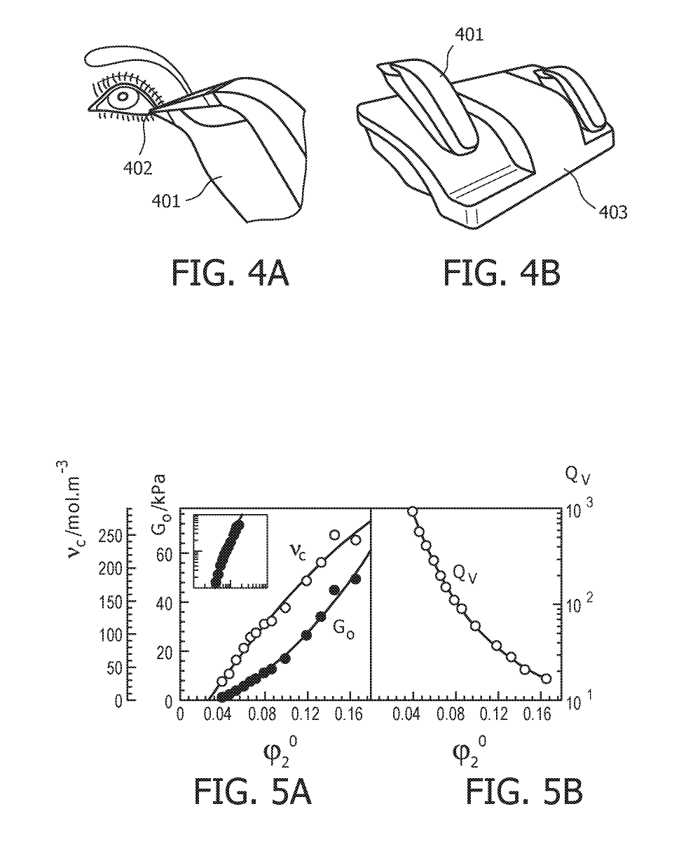

An alternative is to integrate the sampling (micro-) tube in a specific device in which case subsequent analysis can be performed right after sample taking. This option has been implemented in the osmolarity measurement device known as TearLab™ Osmolarity System demonstrated in FIG. 4A, B. A (micro-) tube 402 is integrated in a device 401 mountable in a docking station 403, wherein a tear analysis can be performed using an analysis unit within the docking station 403.

Another practiced method involves placing an absorbing strip of specific ‘filter papers” normally with 7×40 mm dimensions in the lower conjunctiva of the patient's eye after which the patients have to close their eyes for 5 minutes (with the strip in their eye) during which tear fluid is collected. Subsequent to collection the tear fluid has to be isolated before it can be used for analysis. Generally the isolation steps involves measuring the wetted area and its weight, followed by mincing and dissolving in water, elute by centrifuge and repeat the elution step by adding buffer to create specific pH depending on the tear component that needs to be analyzed (e.g. pH 4.5 for such enzymes as lysozomal enzymes).

Tear collection methods known from the past show a number of disadvantages. First, only point measurement is possible: Analysis of tear samples collected by these methods provides “point” data, i.e. data that can be collected only at a specific time point, on tear quality. This does not however provide information about tear composition variability over time. To obtain a baseline or an average value for any measured component therefore, multiple samples need to be obtained at various times if possible; adding to the discomfort and anxiety of the sample collection for the patient. This is especially a problem if the concentration of the compound under analysis is subject to 24 hour variations and/or has a short half time in which case its concentration is a function of time of sample taking.

Another disadvantage includes undesirable effects of eye stimulation: Tt has been shown that stimulating or irritating the eyes to create tear samples causes a marked difference in tear composition resulting in contradictory analysis results. Bringing micro-tubes in contact with the eye surface creates a chance of stimulation by sample collecting tubes that can cause tear generation with a different composition.

Also, methods known from the past show day and night dependency: Tear sample collection during sleeping hours pose more inconvenience to patients and care givers alike hence is not practiced although in some specific cases such as analysis of melatonin levels for determining sleep quality and/or sleep disorder. Samples during sleep are specifically valuable.

A further disadvantage is volume restriction: Under normal conditions each eye contains 7-10 μl of tear. This volume is normally decreased for aging people and more significantly so if they suffer from conditions such as “dry eye”. This naturally imposes a restriction on the available” tear volume for any tear analysis technique that has to rely on a single sample collected for a point measurement.

Contact lenses are acceptable remedies for vision impairment used by millions of people worldwide. Recent introduction of silicone hydrogel contact lenses has been the key for designing therapeutic contact lenses of continuous wear (overnight wear as well for up to 30 days) since they provide significantly higher oxygen permeability avoiding undesired hypoxic side effects.

Recent contact lenses, although having their main application in correction of ametropia can also fulfill requirements for drug delivery over extended periods of time for such applications as relief of post-surgery ocular pain, corneal healing and mechanical protection due to their improved design for trans-corneal penetration as well as drug delivery for an extended period of time. This format therefore does not create anxiety when used for the purpose of tear collection.

This invention proposes to create the contact lens using specifically selected and/or engineered material in such a way that it absorbs and/or collects and retains the ocular fluid in a controlled manner over a specific period of time after which the lens is removed from the eye, the tear fluid extracted from the lens and is subjected to analysis. Some material candidates combined with specific constructs are described in the next section.

Hydrogels are cross-linked polymeric chains that are able to absorb water up to an equilibrium state which causes them to swell in aqueous solutions without dissolving hence retaining their three dimensional (3D) features. The ability of hydrogels to absorb water arises from hydrophilic functional groups attached to the polymer backbone while their resistance to dissolution arises from cross-links between network chains. The equilibrium swelling and the softness (depicted by elastic modulus) of hydrogels depend on the cross link and charge densities of the polymer network as well as on the cross-linked polymer concentration.

This relationship is demonstrated in FIG. 5A, B. In particular, FIG. 5A shows a schematic diagram showing the cross-link density v_{c} and the modulus of elasticity Gₒ of a hydrogel as a function of network concentration φ₂°. FIG. 5B shows the volume swelling ratio Qᵥ of the hydrogel of FIG. 5A as a function of the network concentration φ₂°.

The presence of a cross-linker in the hydrogel matrix therefore, is significant because basic properties of these materials such as definite shape, mechanical strength and transparency are not altered upon hydration.

The characteristics of hydrogels commonly employed in contact lens materials including 2-hydroxyethyl methacrylate (HEMA), methyl methacrylate (MMA) along with N-vinyl pyrrolidone (NVP) and methacrylic acid (MA) determine their physical and chemical properties. Various hydrogels, with different level of water content have been used as contact lens materials due to their softness and moisture content which ensures oxygen permeability which is an important attribute of contact lens materials.

An extended wear contact lens should be able to provide adequate hydrophilicity, as well as oxygen permeability (intrinsic to hydrophobic materials such as polysiloxanes and fluoropolymers), mechanical strength in a hydrated state, compatibility with biological tissues, optical transparency and stability. In the following, some examples for hydrogels, in particular superabsorbent hydrogels, Combined silicone-hydrogel materials and nano-cellulose based hydrogels, are explained, without limiting “hydrogel” to these examples.

Superabsorbent hydrogels (SHs) are slightly cross-linked networks that are able to absorb amounts of aqueous solutions from 10% up to thousands of times their own dry weight. Current studies on the development of SHs have focused on the formulation of highly functional materials with enhanced properties fix suitable applications in different it fields.

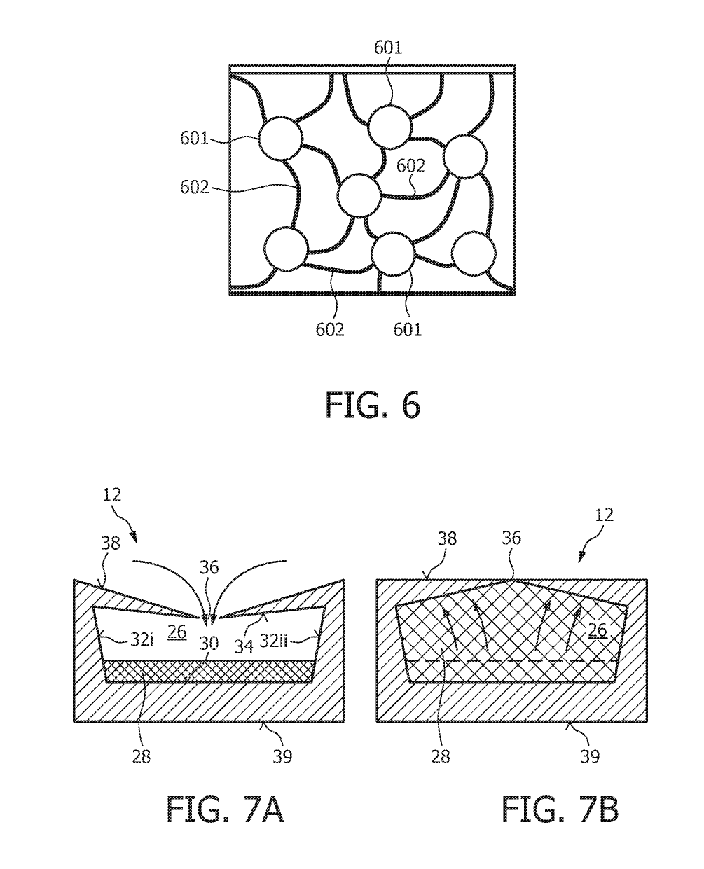

Combined silicone-hydrogel materials are characterized by water permeability as high as conventional hydrogels while at the same time they have significantly higher ion and oxygen permeability. Certain structural parameters can control the properties of hydrogels in terms of permeability and mechanical strength. One such example is shown in FIG. 6 with introduction of network of micro-pores 601 shown as circles connected by chemical bondings 602.

Moreover, materials such as nano-cellulose based hydrogels have been proposed for such applications as wound dressings. This is based on their capability to form 3D self-assembled micro-porous structures that are strongly hydrophilic. Hydrogels may exhibit drastic volume changes in response to specific external stimuli, such as temperature, solvent quality, pH, electric field, etc. Additionally, the surface chemistry can be modified creating strong potential for surface functionalization such as pH sensitivity in a specific environment.

In this context, hydrogel contact lenses with ionic surfaces for example have negative surface charges which facilitate sensitivity to pH as well as attraction to proteins (e.g. lysozyme, a protein present in tear fluid the concentration of which has been shown to have predictive value for dry eye condition).

FIG. 7A shows schematically a modular compartment unit 12 for containing a hydrophilic material 28, in particular hydrogels and/or silicones, in an inner space 26 of the modular compartment unit 12. The inner space 26 is defined by a plurality of inner surfaces of the modular compartment unit 12, in particular a roof surface 34, a bottom surface 30 and a plurality of side surfaces 32i, 32ii.

The modular compartment unit 12 comprises a deformable side 38, which is preferably a top side opposite to the roof surface 34 of the inner space 26. In the open state of the modular compartment unit 12 shown in FIG. 7A, the hydrophilic material 28 has not yet absorbed any ocular fluid, so that its volume remains the same as initially after the hydrophilic material 28 has been introduced into the inner space 26 of the modular compartment unit 12. In particular, as can be seen in FIG. 7A, the initial volume of the hydrophilic material 28 is smaller than the volume of the inner space 26. In this case, there is no mechanical contact between the hydrophilic material 28 and the deformable side 38 of the modular compartment unit 12, so that the deformable side 38 remains in its relaxed state, in which the deformable side 38 is inwardly sunken or inwardly curved.

As can be seen in FIG. 7A, a fluid inlet 36 is formed on the deformable side 38. In particular, the fluid inlet 36 is arranged at a center of the deformable side 38. Due to the own gravity of the deformable side 38, the deformable side 38 is inwardly tilted so that it forms an angle to a rigid side 39 of the modular compartment unit 12 opposite to the deformable side 38. The two arrows pointing from the outside of the modular compartment unit 12 towards the inner space 26 via the fluid inlet 36 indicate that ocular fluid is able to flow into the inner space 26 via the fluid inlet 36.

FIG. 7B shows schematically the modular compartment unit 12 of FIG. 7A in a closed state. In particular, the hydrophilic material 28 has absorbed ocular fluid and expanded in volume. The arrows of FIG. 7B indicate the expansion of the hydrophilic material 28 after having absorbed the ocular fluid. The increase of volume can be clearly seen by the difference between the area showing the hydrophilic material 28 and the area enclosed by the dashed line indicating the initial volume of the hydrophilic material 28. In particular, the hydrophilic material 28 has expanded so that the entire inner space 26 is filled with the hydrophilic material 28. In this state, the deformable side 38 of the modular compartment unit 12 is in direct contact with the hydrophilic material 28, so that the deformable side 38 is supported by the hydrophilic material 28 from below. As a result, the deformable side 38 is not inwardly sunken anymore, but planar, in particular parallel to the rigid side 39 of the modular compartment unit 12. In this way, the fluid inlet 36 is closed so that no ocular fluid can enter the inner space 26 anymore.

Therefore, the volume of the hydrophilic material 28 is restricted by the predetermined maximum volume of the inner space 26 which is reached in the closed state of the modular compartment unit 12 as shown in FIG. 7B. Consequently, the amount of ocular fluid absorbable by the hydrophilic material 28 and thus collectable using the modular compartment unit 12 is predetermined by the initial volume of the hydrophilic material 28 as well as the predetermined maximum volume of the inner space 26. In particular, the predetermined amount of ocular fluid absorbable by the hydrophilic material 28 contained in the modular compartment unit 12 corresponds to the volume difference between the initial volume of the hydrophilic material 28 and the predetermined maximum volume of the inner space 26, as can be seen in FIG. 7A, B.

The modular compartment unit 12 is shown in cross section in FIG. 7A, B. The modular compartment unit 12 can be built in the form of a channel or tube extending in a direction perpendicular to the cross section as shown in FIG. 7A, B. The hydrophilic material 28 can be made out of polymer or membranes such as superabsorbent hydrogels (SHs). In particular, the hydrophilic material 28 can be engineered to absorb the predetermined amount of ocular fluid, as shown above. In this way, the hydrophilic material 28 can be constructed to absorb and retain a predetermined volume of tear fluid over a specific period of time. In particular, the hydrophilic material 28 may have a characteristic diffusion rate for the ocular fluid, so that the time period for absorbing and retaining the predetermined volume of ocular fluid can be derived by dividing the predetermined volume by the diffusion rate. Preferably, diagnostics and drug response monitoring can be performed based on the unobtrusive methods involving at least one modular compartment unit 12 for collecting ocular fluid samples over a selected time period.

During the diffusion of ocular fluid into the hydrophilic material 28, the hydrophilic material 28 swells and expands in volume, until the swelling/volume expansion saturates resulting in a complete filling of the inner space 26 of the modular compartment unit 12 by the hydrophilic material 28 (FIG. 7B).

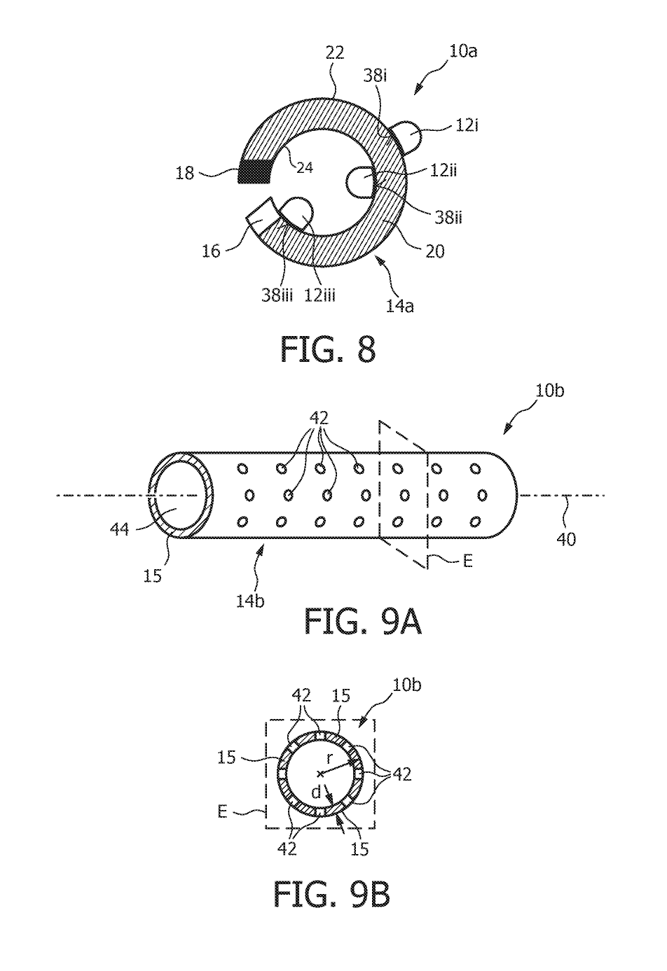

FIG. 8 shows schematically a wearable device 10a comprising a plurality of modular compartment units 12i, 12ii, 12iii, which are detachably connected to a substrate, wherein the substrate is configured as a fluid channel 14a. The fluid channel 14a is a ring-shaped channel comprising an outer edge 22 and an inner edge 24, wherein the inner edge 24 is radially spaced from the outer edge 22 along a first radial direction towards the center of the rings-shaped channel. The fluid channel 14a extends annularly from an open end 16 to a closed end 18 around a revolution axis. The open end 16 is used for introducing ocular fluids into a hollow space 20 within fluid channel 14a. As can be seen in FIG. 8, the ring-shape of the fluid channel 14a covers circumferentially a spherical angle which is smaller than 360′. In the example of FIG. 8, the first radial direction would be contained on the plane defined by the paper while the revolution axis would come out of the paper. The fluid channel 14a may be formed using an inherently hydrophobic material.

The plurality of modular compartment units comprise a first modular compartment unit 12i, which is detachably connected to the outer edge 22 on the exterior of the fluid channel 14a. Further, a second and a third modular compartment unit 12ii, 12iii are detachably connected to the inner edge 24 on the exterior of the fluid channel 14a. The deformable side 38i, 38ii, 38iii of the respective modular compartment unit 12i 12ii, 12iii is arranged to face the outer/inner edge 22, 24 of the fluid channel 14a. In this way, the fluid inlet of modular compartment units 12i, 12ii, 12iii is in fluidic connection with the hollow space 20 of the fluid channel 14a. Ocular fluids entering the hollow space 20 can therefore be collected by the modular compartment units 12i, 12ii, 12iii.

Similar to the mechanism described in conjunction with FIG. 7A, B, the plurality of modular compartment units 12i, 12ii, 12iii functioning as multiple reservoirs connected by the single fluid channel 14a can provide collection of ocular fluids over a specific period of time and in a predetermined volume. For each modular compartment unit 12i, 12ii. 12iii, the time period for the hydrophilic material contained in the respective modular compartment unit 12i, 12ii, 12iii to absorb and retain a predetermined volume of ocular fluid until the respective modular compartment unit 12i, 12ii, 12iii has reached its closed state can be calculated by dividing the predetermined volume over the diffusion rate of the hydrophilic material. The predetermined amount of ocular fluid can, on the other hand, be determined by subtracting the initial volume of the hydrophilic material contained in the respective inner space from the maximum volume of the inner space corresponding to the closed state of the modular compartment unit 12i, 12ii, 12iii.

The wearable device 10a that comprises a plurality of modular compartment units 12i, 12ii, 12iii and the fluid channel 14a is configured to contain a certain volume of hydrophilic materials, for instance SHs, wherein the wearable device 10a can be incorporated into a contact lens. The wearable device 10a can be incorporated in a contact lens. Preferably, the wearable device 10a can be constructed so that the modular compartment units 12i, 12ii, 12iii are arranged peripherally with respect to a central optical section of the contact lens which has one or more vision-related optical requirements. In particular, the wearable device 10a can be constructed so that the central optical section of the contact lens is surrounded by the inner edge 24 of the ring-shaped fluid channel 14a, wherein the central optical section of the contact lens is radially spaced along a first radial direction from the plurality of modular compartment units 12i, 12ii, 12iii. In this way, light incident on the central optical section of the contact lens is not disturbed by the wearable device 10a. Such a peripheral structure can be provided in e.g. a tube format filled with SHs.

The ring-shaped fluid channel 14a can act as tear inlet path in which case it does not need to contain or be filled with a hydrophilic material such as hydrogel. Alternatively, the fluid channel being a ring-shaped tube is configured to contain or be filled with a hydrophilic material such as SHs. In this case, the ring-shaped fluid channel 14a acts itself as a compartment, wherein one or more modular compartment units 12i, ii, iii can be omitted.

In a preferable embodiment, the ring-shaped fluid channel 14a is an integrated part of a contact lens. Various materials compatible with structural and biocompatibility requirements of contact lens can be used.

FIG. 9 shows schematically another wearable device 10b. The wearable device 10b comprises a fluid channel 14b, to which one or more modular compartment units (not shown here) are detachably connectable in a hollow space 44 within the fluid channel 14b. The fluid channel 14b of the device 10b shown in FIG. 9A can be an open ring (i.e., the cylindrical fluid channel 14b can be bent around a revolution axis to adopt a geometry similar to the device illustrated in FIG. 8), wherein the fluid channel 14b is open on one of its two annular ends. Also, the fluid channel 14b may take the form of a “cut ring”, i.e. the fluid channel 14b extends circumferentially over an angle that is smaller than 360°, preferably equal to 180° (“half ring”). In this way, the fluid channel 14b can be in the form of a half tube/channel, i.e. a tube or channel cut in half along its annular axis.

Further, alternatively or additionally to a ring-shaped fluid channel as shown in FIG. 8, the fluid channel 14b of the wearable device shown in FIG. 9A is covered by a porous layer 15 comprising a plurality of pores 42 extending through the layer in a second radial direction perpendicular to an annular axis 40, along which the fluid channel 14b extends. The layer 15 is preferably a membrane layer. In a preferable embodiment, the porous layer 15 covers the “cut ring” both on the plane of the “cut” and at both ends, thereby enabling to control the equilibrium of fluid between the interior and the exterior of the fluid channel 14b. In this case, both ends of the fluid channel 14b are open through the pores 42. Alternatively, one of both ends can be closed off.

In this way, the one or more modular compartment units are encapsulated by the layer 15 of porous material forming the fluid channel 14b. The plurality of pores 42 can be provided in the same or different sizes, wherein the density of the pores can be varied depending on the actual application. In particular, the pore size and density provide a possibility to control tear diffusion: the larger the pore size and/or the density, the higher the diffusion rate of fluids flowing into the hollow space 44 through the pores 42. Pore size values of several micrometers (e.g. 9-13 μm) up to 100 μm can be used. The diameter of the pores may be chosen to be from 1 μm to a few mm.

In the case of a fluid channel with attached one or more modular compartment units as described above, the porous layer 15 may act as a tear fluid inlet. Alternatively, the fluid channel 14b covered by the porous layer 15 forms itself a compartment, in particular a hydrophobic compartment that does not contain or is not filled with hydrophilic material such as hydrogel. The porous layer 15 allows tear fluid diffusion/flow that is regulated/stopped when equilibrium is reached between the outer and the inner side of the porous layer 15.

FIG. 9B shows the wearable device 10b in a cross section indicated by the plane E, wherein the plane E is perpendicular to the annular axis 40 and coincides a plurality of pores 42. The porous layer 15 forming the fluid channel 14b has a thickness of d, wherein the outer radius of the fluid channel 14b is indicated by r. The plurality of pores 42 can be arranged circumferentially with constant or varying distance between adjacent pores 42.

The fluid channel 14a, 14b comprises preferably a hydrophobic material which is inherently hydrophobic, such as silicone, polyester or polyurethane. Further, the one or more modular compartment units 12, 12i, 12ii, 12iii may preferably comprise such inherently hydrophobic material. The modular compartment units are configured to retain the collected ocular fluid samples within the swollen hydrophilic material until the hydrophilic material can be removed from the modular compartment unit. In case of the wearable device 10b shown in FIG. 9, the membrane layer forming the fluid channel 14b can be peeled off when the collection of ocular fluid is completed in order to access the swollen hydrogel.

The present invention therefore provides methods of continuous sample collection for ocular fluids over a specified period of time and known volume using contact lens as a sample collection medium. The tear samples can subsequently be isolated and analyzed to provide an average measure of various compounds in the collected tear fluid. The analysis can aim at detecting biomarkers (e.g. cortisol, melatonin), and/or detecting multiple classes of therapeutic drugs (e.g. phenobarbital, carbamazepine, Methotrexate) as well as determining ocular side-effect of therapeutic drugs (e.g. dry eye).

The present invention further facilitates ocular fluid collection over a specified time period and in minimally invasive and unobtnisive ways. This can be achieved by using the contact lens format as the tear fluid collections means. In particular, the wearable devices 10a, b may be incorporated in a contact lens.

The tear collection approach disclosed herein further enables obtaining biological data averaged over time. Moreover, the proposed method creates less discomfort hence diminishing the anxiety of sample collection experienced in current methods which in some cases (e.g. cortisol) has an adverse influence on the composition of the very compound that the sample is collected for.

Contact lens constructions can be based on hydrophilic materials (e.g. silicone and hydrogels) in which microfluidic compartments are integrated in order to facilitate collection of a pre-defined, specific volume of tear fluid within a specific period of time. Tear fluid can be collected in single or multi-component and/or multi-layered structures as means of time and volume controlled tear collection solutions placed in the eye. A predefined volume of the hydrophilic (e.g. hydrogel) material with specified absorption properties is used to absorb and collect the tear fluid. This construction specifies the completion of tear collection process when swelling is completed (fluid inlet is closed and/or saturation of volume has been reached).

Upon removal of the contact lens to access the tear fluid that is trapped in the hydrogel structure within the contact lens, various means can be employed to collapse the hydrogel structure and isolate the tear fluid for subsequent analysis. The elution step can be similar to extraction of tear fluid sample from filter paper in known tear analysis methods such as (gel) electrophoresis using polyacrylamide that is used for separation of macromolecules based on their size and charge out of tear fluid.

Alternative elution techniques for extracting tear fluid from the hydrogel structure include dissolving the hydrogel-tear fluid system followed by separation or enzymatic digestion of the hydrogel scaffold by e.g. collagenase as well as binding to a specific molecule for enhancing separation (e.g. as in the case of solvent extraction), and/or removing the excess water by means of controlled evaporation. Moreover, various chemical or physical stimuli have been shown to induce a response in the (smart) hydrogel systems. The physical stimuli include temperature, electric filed, light, pressure, sound and magnetic field. The chemical and biochemical stimuli consist of pH and ions as well as specific molecular recognition compounds.

Some of the above-mentioned methods are applied in drug delivery systems involving hydrogels as well. One example is a superporous hydrogels (SPH) containing poly (methacrylic acid-co-acrylamide) that can be synthesized from methacrylic acid and acrylamide through the aqueous solution polymerization, using N, N-methylenebisacrylamide as a crosslinker and ammonium persulfate as an initiator in which a considerable change in swelling can be induced by a change in pH from acidic to basic. This method can therefore be used to extract absorbed tear fluid out of the SPH.

While the invention has been illustrated and described in detail in the drawings and foregoing description, such illustration and description are to be considered illustrative or exemplary and not restrictive; the invention is not limited to the exemplary embodiments disclosed in the present disclosure. Other variations to the disclosed embodiments can be understood and effected by those skilled in the art in practicing the claimed invention, from a study of the drawings, the disclosure, and the appended claims.

In the claims, the word “comprising” does not exclude other elements or steps, and the indefinite article “a” or “an” does not exclude a plurality. A single element or other unit may fulfill the functions of several items recited in the claims. The mere fact that certain measures are recited in mutually different dependent claims does not indicate that a combination of these measures cannot be used to advantage.

A computer program may be stored/distributed on a suitable medium, such as an optical storage medium or a solid-state medium supplied together with or as part of other hardware, but may also be distributed in other forms, such as via the Internet or other wired or wireless telecommunication systems.

The present invention disclosed herein has been described with reference to the preferred embodiments. Modifications and alterations may occur to others upon reading and understanding the preceding detailed description. It is intended that the invention be construed as including all such modifications and alterations insofar as they come within the scope of the appended claims or the equivalents thereof.

Further, as one having ordinary skill in the art shall appreciate in view of the teachings provided herein, features, elements, components, etc. disclosed and described in the present disclosure/specification and/or depicted in the appended Figures may be implemented in various combinations of hardware and software, and provide functions which may be combined in a single element or multiple elements. For example, the functions of the various features, elements, components, etc. shown/illustrated/depicted in the Figures can be provided through the use of dedicated hardware as well as hardware capable of executing software in association with appropriate software. When provided by a processor, the functions can be provided by a single dedicated processor, by a single shared processor, or by a plurality of individual processors, some of which can be shared and/or multiplexed. Moreover, explicit use of the term “processor” or “controller” should not be construed to refer exclusively to hardware capable of executing software, and can implicitly include, without limitation, digital signal processor (“DSP”) hardware, memory (e.g., read only memory (“ROM”) for storing software, random access memory (“RAM”), non-volatile storage, etc.) and virtually any means and/or machine (including hardware, software, firmware, combinations thereof, etc.) which is capable of (and/or configurable) to perform and/or control a process.

Moreover, all statements herein reciting principles, aspects, and exemplary embodiments of the present invention, as well as specific examples thereof, are intended to encompass both structural and functional equivalents thereof. Additionally, it is intended that such equivalents include both currently known equivalents as well as equivalents developed in the future (e.g., any elements developed that can perform the same or substantially similar functionality, regardless of structure). Thus, for example, it will be appreciated by one having ordinary skill in the art in view of the teachings provided herein that any block diagrams presented herein can represent conceptual views of illustrative system components and/or circuitry embodying the principles of the invention. Similarly, one having ordinary skill in the art should appreciate in view of the teachings provided herein that any flow charts, flow diagrams and the like can represent various processes which can be substantially represented in computer readable storage media and so executed by a computer, processor or other device with processing capabilities, whether or not such computer or processor is explicitly shown.

Having described preferred and exemplary embodiments of a wearable device and method for collecting ocular fluid, which exemplary embodiments are intended to be illustrative and not limiting, it is noted that modifications and variations can be made by persons having ordinary skill in the art in view of the teachings provided herein, including the appended FIGS. and claims. It is therefore to be understood that changes can be made into the preferred and exemplary embodiments of the present disclosure which are within the scope of the present invention and exemplary embodiments disclosed and described herein.

Further, it is contemplated that corresponding and/or related systems incorporating and/or implementing the device or such as may be used/implemented in a device in accordance with the present disclosure are also contemplated and considered to be within the scope of the present invention. Moreover, corresponding and/or related method for manufacturing and/or using a device and/or system in accordance with the present disclosure are also contemplated and considered to be within the scope of the present invention.

What is claimed is:

1. A wearable device for collecting ocular fluid of a user, comprising:
a fluid channel configured for enabling a flow of ocular fluid within the fluid channel when the wearable device is worn by the user, the fluid channel having an at least partially tubular cross-section and extending along an annular axis from an open end in an annular geometry around a revolution axis perpendicular to the annular axis, the fluid channel comprising an outer edge and an inner edge spaced from the outer edge along a first radial direction perpendicular to the revolution axis, the open end of the fluid channel being configured to receive an ocular fluid; and at least one of:
a porous layer arranged on an external surface of the fluid channel, the porous layer comprising a plurality of pores extending through the porous layer in a second radial direction perpendicular to the annular axis, wherein the fluid channel contains a hydrophilic material in a hollow space within the fluid channel, the hydrophilic material being provided in a volume that is smaller than the volume of the hollow space within the fluid channel, wherein the hollow space is, when the device is worn by the user, configured to be in fluidic connection with the ocular fluid of the user via the open end and the plurality of pores; or
one or more modular compartment units detachably connected to at least one of the outer edge or the inner edge on an external surface of the fluid channel, wherein the one or more modular compartment units contain a hydrophilic material in an inner space of each modular compartment unit, the hydrophilic material being provided in a volume that is smaller than the volume of the inner space of the corresponding modular compartment unit, and wherein the inner space of each modular compartment unit is, when the device is worn by the user, configured to be in fluidic connection with the ocular fluid of the user via the open end and a corresponding fluid inlet of the modular compartment unit;
wherein at least one of the hydrophilic material contained in the hollow space within the fluid channel or the hydrophilic material contained in the inner space of each modular compartment unit is structured and configured to absorb the ocular fluid and increase in volume in proportion to the amount of ocular fluid absorbed.

2. The device according to claim 1, wherein, when the device comprises the one or more modular compartment units, the fluid inlet is closable by the hydrophilic material contained in the corresponding inner space having absorbed a predetermined amount of ocular fluid.

3. The device according to claim 2, wherein, when the device comprises the one or more modular compartment units, the fluid inlet is formed on a deformable side of the modular compartment unit, the deformable side being inwardly curved before the hydrophilic material contained in the corresponding inner space has absorbed the predetermined amount of ocular fluid.

4. The device according to claim 3, wherein, when the device comprises the one or more modular compartment units, the deformable side of the modular compartment unit is at least one of planar or curved outwardly after the hydrophilic material contained in the corresponding inner space has absorbed the predetermined amount of ocular fluid.

5. The device according to claim 1, wherein, when the device comprises the one or more modular compartment units, the modular compartment units are serially arranged, so that different modular compartment units are configured to collect ocular fluid during different time intervals.

6. The device according to claim 1, wherein, when the device comprises the one or more modular compartment units, the device further comprises one or more additional modular compartment units detachably connected to the fluid channel in a hollow space within the fluid channel.

7. The device according to claim 1, wherein the cross-section of the fluid channel extends circumferentially around the annular axis over an angle that is smaller than 360°.

8. The device according to claim 1, wherein the fluid channel contains the hydrophobic material.

9. The device according to claim 1, wherein the fluid channel extends in the annular geometry between the open end and a closed end.

10. The device according to claim 1, wherein, when the device comprises the porous layer arranged on the external surface of the fluid channel, the porous layer is configured to regulate diffusion of the ocular fluid, wherein the fluid diffusion is stopped when an equilibrium is reached between an outer side of the porous layer and an inner side of the porous layer.

11. The device according to claim 1, wherein, when the device comprises the porous layer arranged on the external surface of the fluid channel, the porous layer comprises a membrane layer.

12. The device according to claim 1, wherein, when the device comprises the one or more modular compartment units, the hydrophilic material contained in the inner space of each modular compartment unit comprises a hydrogel.

13. Device according to claim 1, wherein the hydrophilic material contained in the hollow space within the fluid channel comprises a hydrogel.

14. A method for collecting ocular fluid of a user, using the wearable device according to claim 1, comprising:
using the fluid channel to enable a flow of ocular fluid within the fluid channel when the wearable device is worn by the user, the fluid channel having an at least partially tubular cross-section and extending along an annular axis from an open end in an annular geometry around a revolution axis perpendicular to the annular axis, the fluid channel comprising an outer edge and an inner edge spaced from the outer edge along a first radial direction perpendicular to the revolution axis, the open end of the fluid channel being configured to receive an ocular fluid, wherein the wearable device further includes at least one of:
a porous layer arranged on an external surface of the fluid channel, the porous layer comprising a plurality of pores extending through the porous layer in a second radial direction perpendicular to the annular axis, wherein the fluid channel contains a hydrophilic material in a hollow space within the fluid channel, the hydrophilic material being provided in a volume that is smaller than the volume of the hollow space within the fluid channel, wherein the hollow space is, when the device is worn by the user, configured to be in fluidic connection with the ocular fluid of the user via the open end and the plurality of pores; or
one or more modular compartment units detachably connected to at least one of the outer edge or the inner edge on an external surface of the fluid channel, wherein the one or more modular compartment units contain a hydrophilic material in an inner space of each modular compartment unit, the hydrophilic material being provided in a volume that is smaller than the volume of the inner space of the corresponding modular compartment unit, wherein the inner space of each modular compartment unit is, when the device is worn by the user, configured to be in fluidic connection with the ocular fluid of the user via the open end and a corresponding fluid inlet of the modular compartment unit;
wherein at least one of the hydrophilic material contained in the hollow space within the fluid channel or the hydrophilic material contained in the inner space of each modular compartment unit is structured and configured to absorb the ocular fluid and increase in volume in proportion to the amount of ocular fluid absorbed.

15. A computer program comprising executable program code configured to cause a computer operatively coupled to a wearable device according to claim 1 to carry out the steps of the method according to claim 14 on the wearable device when said computer program is executed on a computer.
